(19) United States
(12) Patent Application Publication
(10) Pub. No.: US 2013/0158350 A1
(43) Pub. Date: Jun. 20, 2013

(54) ELECTRICAL CONNECTING ELEMENT AND ENDOSCOPY SYSTEM

(71) Applicant: Thorsten Juergens, Hamburg (DE)

(72) Inventor: Thorsten Juergens, Hamburg (DE)

(73) Assignee: OLYMPUS WINTER & IBE GMBH, Hamburg (DE)

(21) Appl. No.: 13/818,171

(22) PCT Filed: Jul. 25, 2011
(86) PCT No.: PCT/EP11/03708

§ 371 Date: Feb. 21, 2013

(30) Foreign Application Priority Data
Aug. 25, 2010 (DE) 10 2010 039 731.8

(51) Int. Cl. A61B 1/00; H01R 4/00

(52) U.S. Cl. CPC A61B 1/00124; H01R 4/00
USPC 600/109; 174/74 R

(57) ABSTRACT

An electrical connecting element for supplying current to a light source, particularly an LED light source, that is or can be connected to a side of an endoscope, configured for introducing light into an optical system of the endoscope in a proximal region of the endoscope, and to an endoscopy system having an electrical connecting element, and to a use. Wherein the electrical connecting element is configured as a pre-formed, flat molded body having a longitudinally extended section, wherein the molded body has a curved shape at least in sections and is configured to be plugged onto an endoscope and to clasp tightly clinging thereon, wherein the molded body has electrical lines which have electrical contact surfaces in a proximal end region of the molded body in order to connect to a current supply, and electrical contact surfaces in a distal region for connecting to the light source.

The invention relates to an electrical connecting element for supplying current to a light source, particularly an LED light source, that is or can be connected to a side of an endoscope, the light source being designed for introducing light into an optical system of the endoscope in a proximal region of the endoscope, and to an endoscopy system having an electrical connecting element according to the invention and to a use.

Minimally invasive surgery using endoscopes is performed through a natural or previously created opening in the body through which a longitudinally extended endoscope is introduced into the interior of the body or a body cavity. In doing so, the operator or surgeon cannot directly view the operative field, but depends on an optical system in the endoscope and other optical elements built into the endoscope to introduce light into the interior of the body, and illuminate the operative field and deliver an image of the operative field through the endoscope optically or electronically.

With resectoscopy for example, there is little space available in the endoscope, or respectively resectoscope, so that it is not practical to mount a light source at the distal tip of the endoscope, thus in the region which is introduced into the interior of the body. This is why resectoscopes, and also many other endoscopes, have a connection for a light source in the proximal region at the housing. This can be a connection for fiber optics for an external light source that is not moved together with the endoscope. On the other hand, a current-operated light source can also be disposed on the housing of the endoscope in the proximal region and produce light that is further conducted through an optical system, for example glass fibers, in the interior of a tubular shaft of the endoscope to the distal end.

It is particularly advantageous to connect a separate LED light source to the optical system of the endoscope, because good light intensity can thus be attained, and this retains compatibility with older endoscope product lines and with products or respectively endoscopes from other manufacturers with respect to imaging capabilities.

Such a solution makes handling difficult because the light source must be supplied with current using a cable connection. This can be realized either using a cable leading to an external source of current or the current supply can be realized in a camera head that is connected to the endoscope in the proximal region. A camera head generally has a connection adapter for an ocular funnel of the endoscope, and an optical system by means of which the light exiting from the ocular of the endoscope is focused on a chip, for example a CCD chip.

Based on this prior art, the object of the present invention is to ensure a current supply to a light source that is or can be connected to a side of an endoscope and that guarantees good handling of the endoscope during an operation and a reliable current supply of the light source.

This object is achieved by an electrical connecting element for supplying current to a light source, particularly to an LED light source, that is or can be connected to a side of an endoscope, that is designed to introduce light into an optical system of the endoscope in a proximal region of the endoscope, that is further developed in that the electrical connecting element is designed as a pre-formed, flat molded body having a longitudinally extended section, the molded body having a curved shape at least in sections and being designed to be plugged onto an endoscope and to clasp tightly clinging thereon, the molded body having electrical lines which have electrical contact surfaces in a proximal end region of the molded body for connecting to a current supply, and having electrical contact surfaces in a distal region for connecting to the light source.

The electrical connecting element according to the invention is based on the fundamental idea that a molded part that is or can be adapted to the shape of the main body of the endoscope is used, that is clamped onto the main body of the endoscope as an adapter and due to the shape thereof and the materials used and the elastic properties clamps onto the main body of the endoscope. The adapter, because it clamps and is flat, does not substantially impact the handling of the endoscope. Its longitudinal axial positioning on the endoscope is determined on the one hand by the connection of the electrical contact surfaces on the curved limbs to the corresponding contacts of the light source, and on the other hand by the contacting of the proximal contact surfaces to a current supply. Wires or conductor paths can be used as electrical lines.

In a preferred further development, the molded body opens distally in a bifurcated part with two curved limbs, with which the molded body is or can be clasped tightly clinging to the endoscope, wherein the electrical lines run distally to electrical contact surfaces at the ends of the curved limbs. In this case the molded body clasps the endoscope with the curved limbs so that it cannot slip off the endoscope. Thus, the curved limbs form a section of the molded body which has a curved shape and is designed to be plugged onto an endoscope and to clasp tightly clinging thereon.

Alternatively or additionally to this it is advantageously provided that the molded body is designed at least in sections as a half-pipe, that in cross section in the periphery thereof describes a section of a circle of somewhat more than 180°. This section of a half-pipe is also designed to be plugged onto an endoscope and to clasp tightly clinging thereon.

In one advantageous further development, the proximal end of the molded body is curved hook-shaped in order to be securely clamped to an ocular funnel of the endoscope, wherein the electrical contact surfaces are disposed at the proximal end of the molded body in the hook-shaped curved section, so that an electrical contact to corresponding electrical contact surfaces at an ocular adapter of a camera head is or can be made. Thus, the longitudinal axial fixing of the connecting element to the endoscope is also securely fastened due to the hook-shaped section that is hooked in at the ocular funnel. An ocular adapter of a camera head is designed substantially complementary in shape to the ocular funnel of the endoscope and on the outer surface thereof has electrical contact surfaces which correspond to the contact surfaces of the molded body thereby guaranteeing an electrical current supply to the light source.

Preferably the molded body is designed as an injection molded interconnect device, wherein the molded body is insulated with respect to the endoscope. Such an injection molded interconnect device is also termed a “molded interconnecting device” (MID). These are electronic components with which metal conductor paths are applied on injection molded plastic carriers.

Advantages of MID technology, among others, lie in the extensive design freedom of the components and the integration of electrical and mechanical functions in an injection molded part. Any arbitrary shape can be designed. The plastic of the injection molded connecting element acts as an insulator to the main body of the endoscope and the conducting wires or respectively conductor paths integrated therein form an electrical circuit between the camera head and light source. The conductor paths can also be covered by a further insulating plastic layer or insulating layer of lacquer.

Advantageously the proximal part of the connecting element is designed as a plug-in connector, particularly as a waterproof plug-in connector. Such a plug-in connector can be simply plugged into a corresponding plug contact in the camera head. Further preferably, the ends of the curved limbs on the distal bifurcated part of the molded body are designed to be received in corresponding plug contacts of the light source. This also results in a very simple startup of an endoscope.

An advantageous elasticity is preferably attained in that the molded body is manufactured from an elastic plastic which counters deflections with a spring force. This results in a particularly simple solution for how the molded body can be slid onto the endoscope and clasps thereto.

The object underlying the invention is also achieved by an endoscopy system comprising an endoscope, a light source, particularly an LED light source, that is or can be connected to one side of the endoscope, and a camera head, wherein the endoscope has an optical system and in the proximal region a connector for the light source and an ocular funnel, wherein the camera head has an ocular adapter for connection to the ocular funnel, wherein the ocular adapter has electrical contacts for supplying current to the light source, that is further developed in that for supplying current to the light source an electrical connecting element according to the invention is provided that can be plugged onto the endoscope in the proximal region of the endoscope, and is designed to produce an electrical circuit between the contacts in the ocular adapter of the camera head on one side and the contacts of the light source on the other.

The contacts in the ocular adapter are preferably constructed as concentric annular contacts. As a consequence, the camera head can be rotated with respect to the endoscope without disconnecting the electrical contact which serves for supplying current to the light source.

The object underlying the invention is further solved by the use of an electrical connecting element, particularly as described above, for the electrical current supply to a light source at an endoscope, particularly an endoscopy system according to the invention described above.

The features, advantages and properties named with the electrical connecting element according to the invention, the endoscopy system according to the invention, and for the use according to the invention, apply in each case without restriction also to the respective other subject matters according to the invention. Both the electrical connecting element according to the invention and also the endoscopy system according to the invention and the use have the advantages that an adapter can be easily inserted or respectively attached for producing an electrical connection between a camera head and a pluggable light source for an endoscopic optical system. Due to the shape, the adapter fastens to the endoscope without further auxiliary means. No additional components are necessary in or on the endoscope. Thereby, the endoscope is not more costly despite introducing the lighting, particularly LED lighting.

Depending on the design of the electrical connecting element and the associated endoscope models, a comprehensive compatibility, for instance to prior models or competitor's models, can be attained or also ruled out.

The manufacturing as an injection molded interconnect device is very inexpensive. Here, connecting elements are possible both for single-use, which require no cleaning, as well as designs of electrical connecting elements for multiple use that can be sterilized.

The ocular adapter of the camera head serves as the contact point between the camera head and the connecting element. As a result, the endoscope can be rotated with respect to the camera head without movement taking place relative to the electrical connecting element.

The invention is described below, without restricting the general intent of the invention, based on exemplary embodiments in reference to the drawings, whereby we expressly refer to the drawings with regard to the disclosure of all details according to the invention that are not explained in greater detail in the text. In the figures:

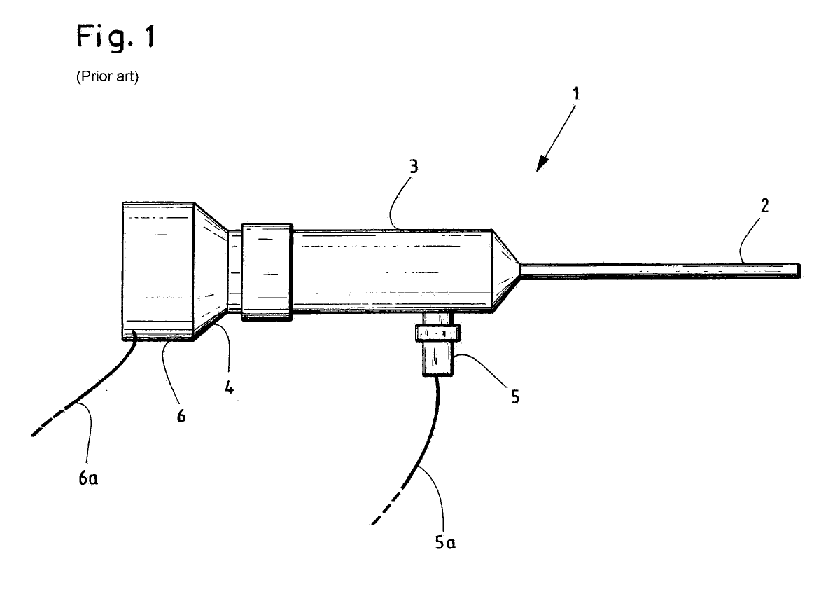

FIG. 1 shows a schematic side view of an endoscope with a known lateral light source,

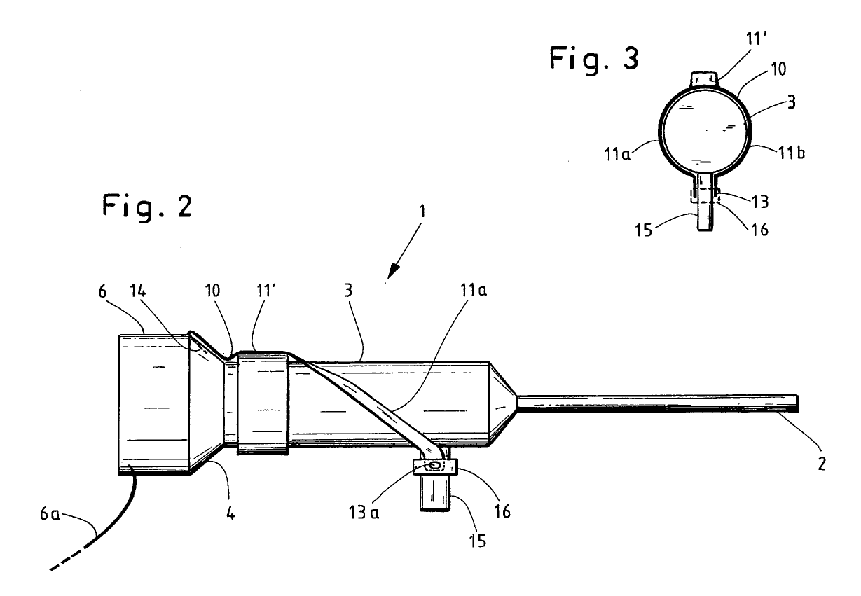

FIG. 2 shows a schematic side representation of an endoscopy system according to the invention,

FIG. 3 shows a schematic representation of a front view of an endoscope according to FIG. 2,

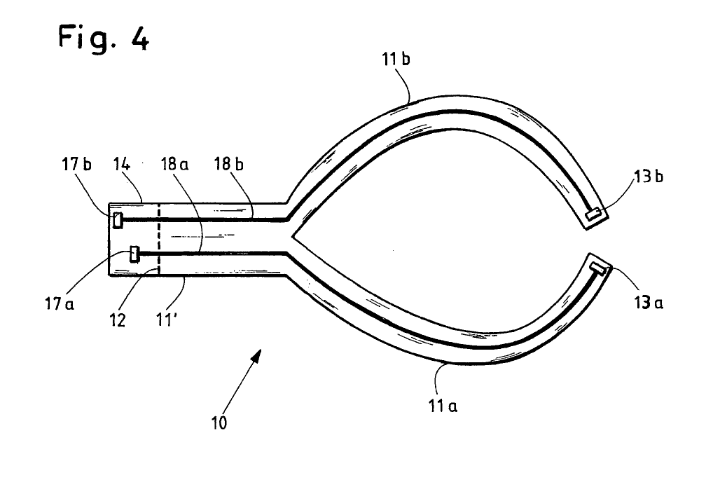

FIG. 4 shows a schematic top view of an electrical connecting element according to the invention, and

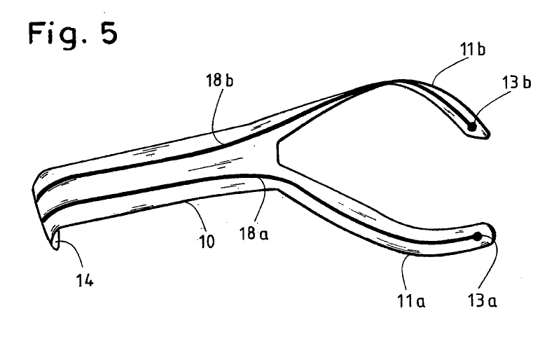

FIG. 5 shows a schematic perspective representation of a connecting element according to the invention.

In the following figures, the same or similar types of elements or respectively corresponding parts are provided with the same reference numbers so that a corresponding re-introduction can be omitted.

FIG. 1 shows a schematic side view of an endoscopy system according to the prior art. This comprises an endoscope 1 that at the distal end has a tubular shaft 2 with an optical system. During minimally invasive surgery or a minimally invasive examination, this tubular shaft 2 is introduced through an opening in the body into a body cavity. The tubular shaft 2 leads into a housing 3 that in turn, on the proximal end, that is, on the end which is disposed toward the surgeon or operator, leads into an ocular funnel 4 having an ocular, not shown. The housing 3 also serves for handling the endoscope 1.

On the side of the housing 3 of the endoscope 1, a light source 5, particularly an LED light source, is disposed which introduces bright light from the side into the optical system of the endoscope 1, from where the introduced light exits at the distal end, that is, at the tip of the tubular shaft 2 in order to illuminate an operative field. The light source 5 has a connection cable 5a. In the case of a customary optical system, the light source 5 can be an adapter to which a glass fiber bundle is attached as a connection cable 5a. The light delivered through the glass fiber bundle is then introduced into the endoscope 1 by means of the adapter. In an alternative version, there is an active light source 5, for example on the basis of LEDs, halogen luminaries or the like, wherein in this case, the connection cable 5a is a current supplying cable.

A schematically represented camera head 6 having an ocular adapter, not shown, is disposed at the ocular funnel 4 of the endoscope 1 and captures the light exiting from the ocular of the endoscope 1 using its own optical system, and focuses the light on an optical image sensor, for example, a CCD chip. By means of the connector 6a for the camera head 6, the camera head 6 is supplied with current, image signals from the area sensor are transferred to an external evaluation unit, and control signals are transferred to the camera head 6.

The endoscope 1 according to FIG. 1 is relatively complicated to handle because two different connection cables 5a for the light source and 6a for the camera head are present, both of which limit the movement of the operator.

FIG. 2 shows a schematic side view of an endoscopy system according to the invention that differs from the endoscopy system according to FIG. 1 in that the light source 5 is not externally supplied with current using a connection cable 5a, but rather via a connecting element 10 that is implemented as a flat molded body. For this purpose the camera head 6 has contact points for a current supply for a light source 15. The light source 15 has corresponding contact points and a connection nut 16, to which corresponding contact points 13a, 13b of the connecting element 10 can be clamped and contacted. For the purpose of clarity, no conductor paths or electrically conducting wires are shown in the FIGS. 2 and 3.

The connecting element 10 has a proximal longitudinally extended section 11′ that at the location of the ocular funnel 4 in a section 14 is curved hook-shaped. Thus, the connecting body 10 is secured against slipping in the direction of the distal end of the endoscope 1. Behind the longitudinally extended section 11′, the connecting element 10 bifurcates into two curved limbs 11a, 11b, of which in FIG. 2 only one limb 11a is shown. The limb 11b is obscured by the endoscope 1.

It can be seen in FIG. 3, based on a frontal view of the endoscope 1 or respectively the housing 3 of the endoscope 1, that the housing 3 has a cylindrical cross-section. The connecting element 10 with the limbs 11a and 11b thereof securely grasps around the housing 3 and is securely clamped thereto. Due to the flexibility of the materials used, the connecting element 10 in FIG. 3 can be pulled off upward from the housing 3. The longitudinally extended section 11′ of the connecting element 10 that sits on an ocular funnel, not shown, and hooks in, is also represented.

The light source 15 is represented at the lower end of the housing 3. The limbs 11a and 11b open at this location into two sections which are each guided along on a side of the light source 15 and with the ends thereof provided with contact surfaces, ending in the region of the light source 15, which has corresponding contact points. The connection is secured by means of a connection nut 16, which can be implemented also as a connection element having plug contacts. An appropriate nut 16 can be equipped as a threaded nut or a nut having a bayonet connection, or with another securing mechanism.

Considering FIG. 2 again, it can be seen that the end sections 11a, 11b are guided laterally at the light source 15 into the connection nut 16. A contact surface 13a of the limb 11a is also shown at this location.

FIG. 4 shows a schematic top view of the flat molded body of the connecting element 10. The proximal section consists of a longitudinally extended section 11′ that on the far side of a kink, which is represented by a dotted line, is bent in order to be securely clamped to an ocular funnel 4. In the direction of the distal end, the connecting element 10 bifurcates into two curved limbs 11a and 11b, which are disposed about the housing of an endoscope in order to securely clamp the connecting element 10 to the housing 3.

The connecting element 10 has conductor paths 18a, 18b which are guided in parallel in the longitudinally extended section 11′, and are respectively divided into the curved limbs 11a and 11b. In the proximal region, the conductor paths 18a, 18b end in contact surfaces 17a, 17b which contact corresponding contacts of a connection adapter of a camera head 6. At the distal end, the conductor paths 18a, 18b end in contact surfaces 13a, 13b which contact corresponding contact points at a light source 15.

The proximal contact surfaces 17a and 17b are offset with respect to each other so that it is possible to use a camera head having concentric contact circles or slip rings such that the endoscope 1 can be rotated arbitrarily using the camera head 6 without disconnecting the supply of current to the light source 15. The contact points 17a and 17b have a sliding contact to the respective slip rings in the camera head 6.

FIG. 5 shows a schematic perspective representation of a connecting element 10 according to the invention in which it can be seen that a hook-shaped section 14 is provided at the proximal end, and the curved limbs 11a, 11b are shaped so that they clasp a cylindrical endoscope housing.

All named features, including those taken from the drawings alone, and individual features, which are disclosed in combination with other features, are considered individually and in combination as essential to the invention. Embodiments according to the invention can be fulfilled through individual characteristics or a combination of several characteristics.

### REFERENCE LIST

2 endoscope
2 tubular shaft with optical system
3 housing
4 ocular funnel
5 light source
5a connection cable for the light source
6 camera head
6a connection for the camera head
10 connecting element
11a, 11b curved limb
11′ longitudinally extended section
12 kink
13a, 13b contact surface
14 hook-shaped section
15 LED light source
16 connection nut
17a, 17b contact surface
18a, 18b conductor path

1. An electrical connecting element for supplying current to a light source connected to a side of an endoscope, for introducing light into an optical system of the endoscope in a proximal region of the endoscope, wherein the electrical connecting element is configured as a pre-formed, flat molded body having a longitudinally extended section, wherein the molded body has a curved shape at least in sections, and is configured to be plugged onto an endoscope and to clasp tightly clinging thereon, wherein the molded body has electrical lines, which have electrical contact surfaces in a proximal end region of the molded body for connecting to a current supply, and have electrical contact surfaces in a distal region for connecting to the light source.

2. The electrical connecting element according to claim 1, wherein the molded body distally opens into a bifurcated part having two curved limbs with which the endoscope is clasped tightly clinging, wherein the electrical lines run distally to electrical contact surfaces at the ends of the curved limbs.

3. The electrical connecting element according to claim 1, wherein the molded body is configured at least in sections as a half-pipe that in the cross section of the periphery thereof describes a segment of a circle of somewhat more than 180°.

4. The electrical connecting element according to claim 1, wherein the proximal end of the molded body is curved hook-shaped, in order to be securely clamped to an ocular funnel of the endoscope, wherein the electrical contact surfaces are disposed at the proximal end of the molded body in the curved hook-shaped section, so that an electrical contact is made to corresponding electrical contact surfaces at an ocular adapter of a camera head.

5. The electrical connecting element according to claim 1, wherein the molded body is configured as an injection molded interconnect device, wherein the molded body is insulated with respect to the endoscope.

6. The electrical connecting element according to claim 1, wherein the proximal part of the connecting element is configured as a, particularly waterproof, plug-in connector.

7. The electrical connecting element according to claim 2, wherein the ends of the curved limbs of the distal bifurcated part of the molded body are to be received in corresponding plug contacts of the light source.

8. The electrical connecting element according to claim 1, wherein the molded body is produced from an elastic plastic which counters deflections with spring force.

9. An endoscopy system comprising:
an endoscope,
a light source that is connected to a side of the endoscope, and
a camera head,
wherein the endoscope has an optical system and in the proximal region a connector for the light source and an ocular funnel,
wherein the camera head has an ocular adapter for connection to the ocular funnel, wherein the ocular adapter has electrical contacts for supplying current to the light source, wherein, for supplying current to the light source an electrical connecting element according to claim 1 is provided that is plugged onto the endoscope in the proximal region of the endoscope, and is configured to produce an electrical circuit between the contacts in the ocular adapter of the camera head on the one hand and the contacts of the light source on the other.

10. The endoscopy system according to claim 9, wherein the contacts in the ocular adapter are configured as concentric annular contacts.

11. A use of an electrical connecting element according to claim 1, for supplying electrical current to a light source at an endoscope.

12. The use of an electrical connecting element of claim 11, further comprising an endoscopy system comprising:
an endoscope,
a light source that is connected to a side of the endoscope, and
a camera head,
wherein the endoscope has an optical system and in the proximal region a connector for the light source and an ocular funnel,
wherein the camera head has an ocular adapter for connection to the ocular funnel, wherein the ocular adapter has electrical contacts for supplying current to the light source, wherein, for supplying current to the light source the electrical connecting element is provided that is plugged onto the endoscope in the proximal region of the endoscope, and is configured to produce an electrical circuit between the contacts in the ocular adapter of the camera head on the one hand and the contacts of the light source on the other.

13. The electrical connecting element according to claim 1, wherein the light source is an LED light source.
